(19) United States
(12) Patent Application Publication
(10) Pub. No.: US 2026/0071027 A1
(43) Pub. Date: Mar. 12, 2026

(54) NEAR INFRARED AND RED LIGHT ABSORBING COMPOSITE RESIN PARTICLES

(71) Applicant: Agfa-Gevaert NV, Mortsel (BE)

(72) Inventors: Viktor Merz, Mortsel (BE); Johan Loccufier, Mortsel (BE)

(73) Assignee: Agfa-Gevaert NV, Mortsel (BE)

(21) Appl. No.: 19/105,238

(22) PCT Filed: Aug. 18, 2023
(86) PCT No.: PCT/EP2023/072759

§ 371 Date: Feb. 20, 2025

(30) Foreign Application Priority Data
Aug. 22, 2022 (EP) 22191378.3

(51) Int. Cl. C08G 69/10; A61N 5/06; A61N 5/067; C08K 5/11; C08K 5/20; C08K 5/3415; C08K 5/3445; C08K 5/353; C08K 5/46; G01N 21/64

(52) U.S. Cl. CPC C08G 69/10; A61N 5/062; A61N 5/0625; A61N 5/067; C08K 5/11; C08K 5/20; C08K 5/3415; C08K 5/3445; C08K 5/353; C08K 5/46; G01N 21/6428; A61N 2005/0659; G01N 2021/6439

(57) ABSTRACT

A composite resin particle comprising a dye and a resin selected from the group consisting of poly(amino acids), polyphosphazenes, polysaccharide derivatives, poly(esters), poly(ortho esters), poly(cyano-acrylates) and copolymers thereof, characterized in that the composite resin particle has an absorption maximum in a range from 600 nm to 750 nm and that the average particle size of the particles is between 0.03 μm and 3 μm. The composite resin particles are suitable for opto-medical applications such as phototherapies including photothermal therapy (PTT), photodynamic therapy (PDT), chemo-photodynamic nanotherapeutics and fluorescence medical imaging.

### TECHNICAL FIELD

The present invention relates to a biocompatible organic nano- and microparticle design for opto-medical applications such as phototherapies including photothermal therapy (PTT), photodynamic therapy (PDT), chemo-photodynamic therapy and fluorescence medical imaging.

### BACKGROUND ART

Red and near infrared (NIR) laser technology is gaining importance in non-invasive treatment of different diseases and in medical diagnostics, including photothermal therapy, photodynamic therapy, fluorescence imaging and photo-acoustic imaging. Several of these technologies rely on NIR absorbing nanoparticles, where often inorganic nanoparticles are used, such as gold nanomaterials, carbon nanomaterials, including carbon nanotubes, metal sulfides, metal oxides and different upconverting nanoparticles.

The use of inorganic optothermal converting nanoparticles has extensively been reviewed (Raza et al., Journal of Materials Research and Technology, 8 (1), 1497-1509 (2019); Wang et al., International Journal of Nanomedicine, 15, 1903-1914 (2020)). Although giving excellent NIR response, these nanoparticles are not biodegradable and hold the risk for bioaccumulation and long retention time in the body that could potentially increase their probability of long term toxicity. Therefore, nanoparticles based on biocompatible organic NIR absorbers would be highly preferred.

Although several classical organic NIR absorbers are well documented in opto-medical applications, cyanine dyes are a particularly preferred class of NIR absorbers (hereafter denoted as NIR-absorbers) due to their high molar extinction coefficient. High molar extinction of NIR laser light has the advantage that the amount of required NIR absorber can be reduced and makes application of in vitro and in vivo imaging and treatment of deeply-sited diseases such as tumours, possible. One of the best known and also FDA approved cyanine dye, is indocyanine green (ICG).

NIR absorbers such as indocyanine green have to be integrated in nanoparticles to increase their lifetime in the body, be dispersible in the aqueous fluids of the body, prevent photo-bleaching and increase its tumour targeting ability.

US2021154335A discloses indocyanine green in PEI resin nanoparticles.

Zeng et al. in Mol. Pharmaceutics 2012, 514-522 discloses nanoparticles of ICG which forms J-aggregates and which are encapsulated by PL-PEG-mAB or PL-PEG polymers.

Several designs of nanoparticles have been proposed in the prior art and recently reviewed (Zhu et al. Biomater. Sci, 6, 746-765 (2018); Zhu et al., Current Medicinal Chemistry, 26, 1389-1405 (2019); Ng. Et al., Chemical reviews, 115, 11012-11042 (2015); Shao et al., RSC Nanoscience & Nanotechnology, 40, 125-157 (2016)). Proposed nanoparticle designs often require laborious synthetic protocols, hampering easy tuning of the nanoparticles towards different applications. Therefore, there is a need for cyanine dye containing nanoparticles that are accessible via simple and scalable industrial technologies. One of these simple and scalable industrial technologies, is solvent evaporation.

Cyanine dyes, such as indocyanine green are however, often not compatible with industrial and easy scalable technologies such as solvent evaporation to physically integrate them into biocompatible nanoparticles. Indocyanine green is not or very limited soluble in the solvents currently used in solvent evaporation techniques. Due to its partial water solubility and degradation in water, the indocyanin green dyes have to be completely encapsulated to avoid any contact with water. This requirement further limits the availability of techniques for making fully encapsulated indocyanin green nano- and microparticles.

The encapsulation of NIR absorbing dye, however, mostly initiates a spectral shift towards longer wavelengths. Due to the position of the peak absorbance of a lot of cyanine dyes and due to the red-shift towards higher wavelengths as a consequence of the encapsulation, a lot of dyes known in such as IR700, are not sensitive to light from red lasers having an emission wavelength between 600 and 750 nm.

Besides NIR lasers, these red lasers represent a considerable installed base of imaging sources in phototherapies and fluorescence medical imaging. Therefore, dyes having an absorption wavelength between 580 nm and 750 nm have to be encapsulated to obtain nanoparticles suitable for phototherapies and fluorescence imaging by means of red lasers and NIR-lasers.

Hence, there is a need to integrate Red and Near Infrared (hereafter denoted as R/NIR) absorbing dyes having an absorption maximum from 580 nm to 750 nm with a high molar extinction coefficient in biocompatible nanoparticles, using industrial solvent evaporation technologies.

### SUMMARY OF INVENTION

It is an object of the invention to provide a nano- or microcapsule of biocompatible polymers containing specific R/NIR absorbers as defined in claim 1.

It is a further aspect of the present invention to provide an aqueous dispersion of the composite resin particles as defined in claim 1. The aqueous dispersion is defined in claim 11.

According to another aspect, the present invention includes an industrial scalable method of integrating the specific class of R/NIR absorbers with biocompatible polymers as defined in claim 16.

Other features, elements, steps, characteristics and advantages of the present invention will become more apparent from the following detailed description of preferred embodiments of the present invention. Specific embodiments of the invention are also defined in the dependent claims.

### DESCRIPTION OF EMBODIMENTS

The objects of the present invention are realized by composite resin particles comprising a biocompatible resin and a dye, characterized in that the resin particle has an absorption maximum from 600 nm to 750 nm, more preferably from 600 nm to 700 nm and most preferably from 610 nm to 650 nm, and that the average particle size of the particles is between 0.03 μm and 3 μm. The absorption maximum of the composite resin particle is to be measured on an aqueous dispersion of the composite resin particle (at a concentration of 0.19 mg/mL).

### A. THE COMPOSITE RESIN PARTICLE

### A.1. The Dye

The dye present in the composite resin particle according to the present invention can be selected from any known class of dyes being suitable to achieve an absorption maximum from 600 nm to 750 nm, more preferably from 600 nm to 700 nm and most preferably from 610 nm to 650 nm when incorporated in a composite resin particle. Typical classes of dyes are selected from the classes disclosed in Industrial Dyes, Chemistry, Properties, Applications edited by Klaus Hunger (Wiley-VCH, ISBN 3-527-30426-6), Color Chemistry, Syntheses, Properties and Applications of Organic Dyes and Pigments (Heinrich Zollinger, VCH, ISBN 0-89573-421-4) and The Chemistry and Application of Dyes, edited by David R. Waring and Geoffrey Hallas (Plenum Press, ISBN 0-306-43278-1).

Practically, it seems that the dye has preferably an absorption maximum from 580 nm to 750 nm.

The dyes can be selected from the group consisting of azo-dyes, phtalocyanines, anthraquinones, indigoid dyes, cyanines, merocyanines, oxonoles, triaryl methane dyes, azomethine dyes and indoaniline dyes. Cyanine dyes are particularly preferred.

In a first particularly preferred embodiment, the dye according to the present invention is a squarilium dye according to general formula I

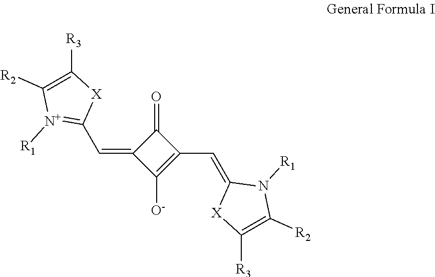

wherein
R₁ is selected from the group consisting of a substituted or unsubstituted alkyl group, a substituted or unsubstituted alkenyl group, a substituted or unsubstituted alkynyl group, a substituted or unsubstituted alkaryl group, a substituted or unsubstituted aralkyl group and a substituted or unsubstituted aryl or heteroaryl group.
R₂ and R₃ are independently selected from the group consisting of a hydrogen, a substituted or unsubstituted alkyl group, a substituted or unsubstituted alkenyl group, a substituted or unsubstituted alkynyl group, a substituted or unsubstituted alkaryl group, a substituted or unsubstituted aralkyl group, a substituted or unsubstituted aryl or heteroaryl group, a halogen, an ester, an amide, an ether, a nitro, a nitrile, an amine a ketone and an aldehyde
R₂ and R₃ may represent the necessary atoms to form a five or six membered ring
X is selected from the group consisting of S, O, NR₄ and CR₅R₆
R₄, R₅ and R₆ are independently selected from the group consisting of a substituted or unsubstituted alkyl group, a substituted or unsubstituted alkenyl group, a substituted or unsubstituted alkynyl group a substituted or unsubstituted alkaryl group, a substituted or unsubstituted aralkyl group and a substituted or unsubstituted aryl or heteroaryl group.
R₅ and R₆ may represent the necessary atoms to form a five to eight membered ring.

In a further preferred embodiment, R₂ and R₃ represent the necessary atoms to form a six membered aromatic ring, optionally further annulated with an additional aromatic ring.

In an even further preferred embodiment, R₁ and R₄ are selected from the group consisting of a substituted or unsubstituted alkyl group, a substituted or unsubstituted alkenyl group and a substituted or unsubstituted alkynyl group, a substituted or unsubstituted alkyl group being more preferred. In an even further preferred embodiment, R₅ and R₆ independently represent a substituted or unsubstituted alkyl group, an unsubstituted alkyl group being more preferred, a methyl group being the most preferred. In a particularly preferred embodiment, R1 and R4 represent a substituted or unsubstituted branched alkyl group. A branched alkyl chain is defined as an alkyl chain comprising at least one secondary or tertiary carbon atom.

Typical examples of squarilium dyes according to the present invention are given in Table 1, without being limited thereto.

TABLE 1

| | |
|---|---|
| 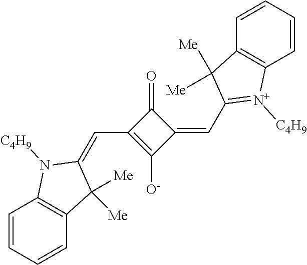 | SQ-1 |
| 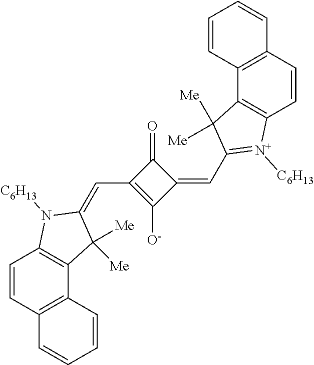 | SQ-2 |
| 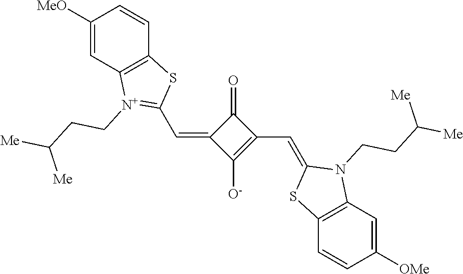 [SMILES: COc1ccc2c(c1)N(CCC(C)C)/C(=C/C1=C([O-])C(=C/c3sc4ccc(OC)cc4[n+]3CCC(C)C)/C1=O)S2] | SQ-3 |
| 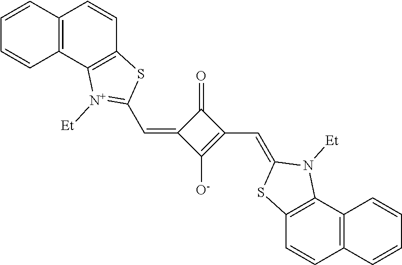 [SMILES: CCN1/C(=C/C2=C([O-])C(=C/c3sc4ccc5ccccc5c4[n+]3CC)/C2=O)Sc2ccc3ccccc3c21] | SQ-4 |
| 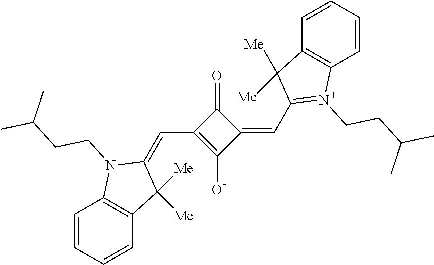 | SQ-5 |
| 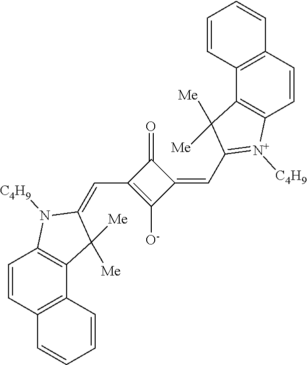 | SQ-6 |

In a further preferred embodiment, the dye present in the composite resin particle according to the present invention is a pentamethine cyanine dye according to general formula II

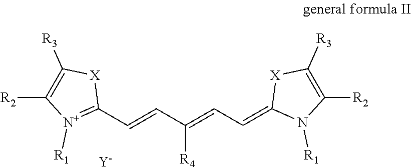

wherein
R₁ is selected from the group consisting of a substituted or unsubstituted alkyl group, a substituted or unsubstituted alkenyl group, a substituted or unsubstituted alkynyl group a substituted or unsubstituted alkaryl group, a substituted or unsubstituted aralkyl group and a substituted or unsubstituted aryl or heteroaryl group.
R₂ and R₃ are independently selected from the group consisting of a hydrogen, a substituted or unsubstituted alkyl group, a substituted or unsubstituted alkenyl group, a substituted or unsubstituted alkynyl group, a substituted or unsubstituted alkaryl group, a substituted or unsubstituted aralkyl group, a substituted or unsubstituted aryl or heteroaryl group, a halogen, an ester, an amide, an ether, a nitro, a nitrile, an amine a ketone and an aldehyde
R₂ and R₃ may represent the necessary atoms to form a five or six membered ring
R₄ is selected from the group consisting of a hydrogen, a a substituted or unsubstituted alkenyl group, a substituted or unsubstituted alkynyl group a substituted or unsubstituted alkaryl group, a substituted or unsubstituted aralkyl group and a substituted or unsubstituted aryl or heteroaryl group.
X is selected from the group consisting of S, O, NR₅ and CR₆R₇
R₅, R₆ and R₇ are independently selected from the group consisting of a substituted or unsubstituted alkyl group, a substituted or unsubstituted alkenyl group, a substituted or unsubstituted alkynyl group a substituted or unsubstituted alkaryl group, a substituted or unsubstituted aralkyl group and a substituted or unsubstituted aryl or heteroaryl group.
R₆ and R₇ may represent the necessary atoms to form a five to eight membered ring.

In a further preferred embodiment, R₂ and R₃ represent the necessary atoms to form a six membered aromatic ring, optionally further annulated with an additional aromatic ring.

In an even further preferred embodiment, R₁ and R₅ are selected from the group consisting of a substituted or unsubstituted alkyl group, a substituted or unsubstituted alkenyl group and a substituted or unsubstituted alkynyl group, a substituted or unsubstituted alkyl group being more preferred. In an even further preferred embodiment, R₆ and R₇ independently represent a substituted or unsubstituted alkyl group, an unsubstituted alkyl group being more preferred, a methyl group being the most preferred. In a particularly preferred embodiment, R₁ and R₅ represent a substituted or unsubstituted branched alkyl group. In another preferred embodiment R4 is selected from the group consisting of a hydrogen and a substituted or unsubstituted alkyl group, a hydrogen and a lower alkyl group being more preferred, a hydrogen being the most preferred.

Typical pentamethine cyanine dyes according to general formula II are given in Table 2 without being limited thereto.

TABLE 2

| | |
|---|---|
| 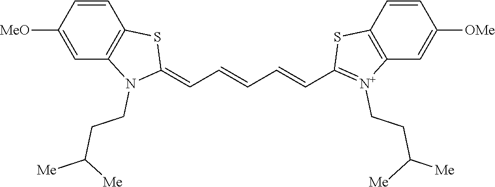 [SMILES: COc1ccc2c(c1)N(CCC(C)C)/C(=C/C=C/C=C/c1sc3ccc(OC)cc3[n+]1CCC(C)C)S2] 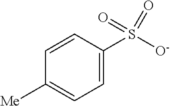 [SMILES: Cc1ccc(S(=O)(=O)[O-])cc1] | PC-1 |
| 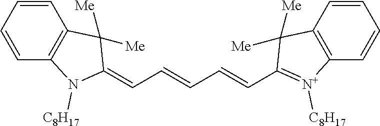 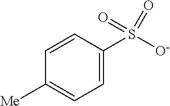 [SMILES: Cc1ccc(S(=O)(=O)[O-])cc1] | PC-2 |
| 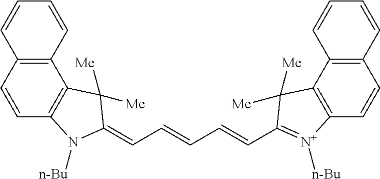 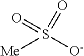 | PC-3 |
| 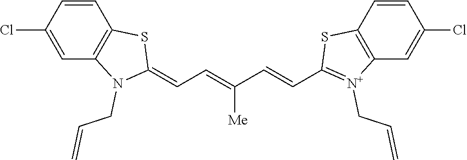 [SMILES: C=CCN1/C(=C/C=C(C)/C=C/c2sc3ccc(Cl)cc3[n+]2CC=C)Sc2ccc(Cl)cc21] 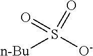 | PC-4 |
| 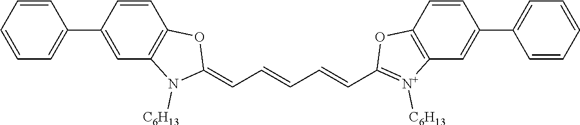 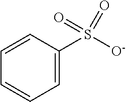 | PC-5 |
| 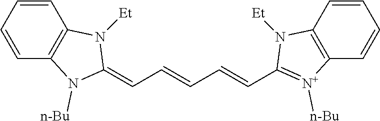 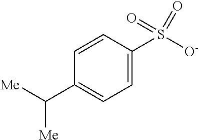 [SMILES: CC(C)c1ccc(S(=O)(=O)[O-])cc1] | PC-6 |

In the most preferred embodiment, the dye present in the composite resin particle according to the present invention is a cyanine dye according to general formula III.

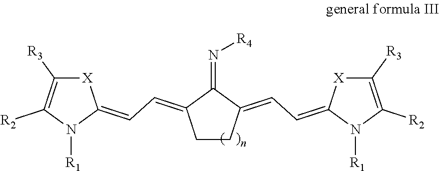

wherein
R₁ is selected from the group consisting of a substituted or unsubstituted alkyl group, a substituted or unsubstituted alkenyl group, a substituted or unsubstituted alkynyl group a substituted or unsubstituted alkaryl group, a substituted or unsubstituted aralkyl group and a substituted or unsubstituted aryl or heteroaryl group.
R₂ and R₃ are independently selected from the group consisting of a hydrogen, a substituted or unsubstituted alkyl group, a substituted or unsubstituted alkenyl group, a substituted or unsubstituted alkynyl group, a substituted or unsubstituted alkaryl group, a substituted or unsubstituted aralkyl group, a substituted or unsubstituted aryl or heteroaryl group, a halogen, an ester, an amide, an ether, a nitro, a nitrile, an amine a ketone and an aldehyde (check)
R₂ and R₃ may represent the necessary atoms to form a five or six membered ring
R₄ is selected from the group consisting of a substituted or unsubstituted alkyl group, a substituted or unsubstituted alkenyl group, a substituted or unsubstituted alkynyl group, a substituted or unsubstituted alkaryl group, a substituted or unsubstituted aralkyl group and a substituted or unsubstituted aryl or heteroaryl group.
X is selected from the group consisting of S, O, NR₅ and CR₆R₇
R₅, R₆ and R₇ are independently selected from the group consisting of a substituted or unsubstituted alkyl group, a substituted or unsubstituted alkenyl group, a substituted or unsubstituted alkynyl group, a substituted or unsubstituted alkaryl group, a substituted or unsubstituted aralkyl group and a substituted or unsubstituted aryl or heteroaryl group.
R₆ and R₇ may represent the necessary atoms to form a five to eight membered ring.

In a further preferred embodiment, R₂ and R₃ represent the necessary atoms to form a six membered aromatic ring, optionally further annulated with an additional aromatic ring.

In an even further preferred embodiment, R₁ and R₅ are selected from the group consisting of a substituted or unsubstituted alkyl group, a substituted or unsubstituted alkenyl group and a substituted or unsubstituted alkynyl group, a substituted or unsubstituted alkyl group being more preferred.

In an even further preferred embodiment, R₆ and R₇ independently represent a substituted or unsubstituted alkyl group, an unsubstituted alkyl group being more preferred, a methyl group being the most preferred. In the most preferred embodiment, R₄ is selected from the group consisting of a substituted or unsubstituted alkyl group, a substituted or unsubstituted alkenyl group and a substituted or unsubstituted alkenyl group, a substituted or unsubstituted alkyl group being more preferred. In a particularly preferred embodiment, R₁, R₄ and R₅ represent a substituted or unsubstituted branched alkyl group.

Typical examples of cyanine dyes according to general formula III are given in Table 3 without being limited thereto.

TABLE 3

| | |
|---|---|
| 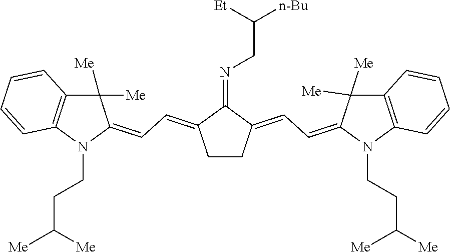 | AC-1 |
| 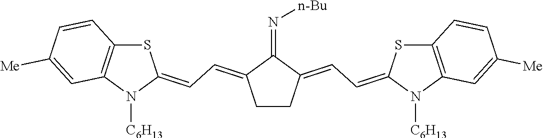 | AC-2 |
| 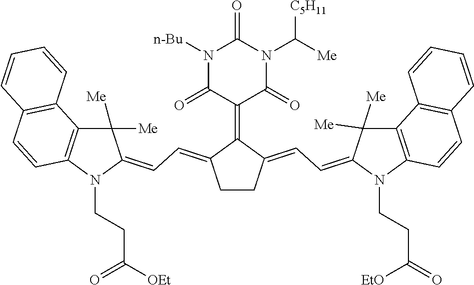 | AC-3 |
| 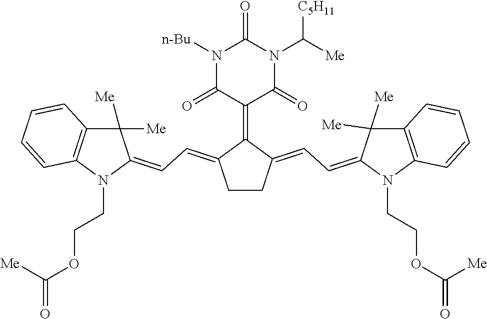 | AC-4 |

Examples of other embodiments according to the present invention are given in Table 4, without being limited thereto.

TABLE 4

| | |
|---|---|
| 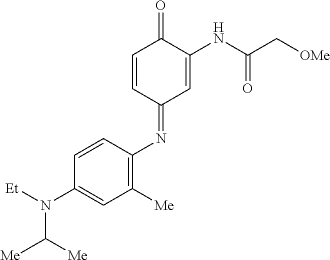 [SMILES: CCN(c1ccc(/N=C2\C=CC(=O)C(NC(=O)COC)=C2)c(C)c1)C(C)C] | DY-1 |
| 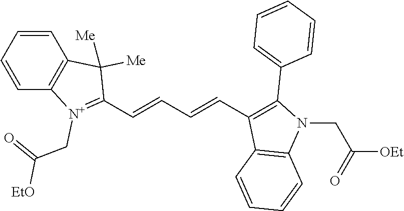 [SMILES: CCOC(=O)Cn1c(-c2ccccc2)c(/C=C/C=C/C2=[N+](CC(=O)OCC)c3ccccc3C2(C)C)c2ccccc21] 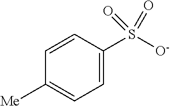 [SMILES: Cc1ccc(S(=O)(=O)[O-])cc1] | DY-2 |
| 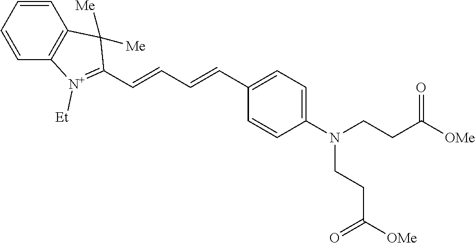 [SMILES: CC[N+]1=C(/C=C/C=C/c2ccc(N(CCC(=O)OC)CCC(=O)OC)cc2)C(C)(C)c2ccccc21] 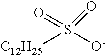 | DY-3 |
| 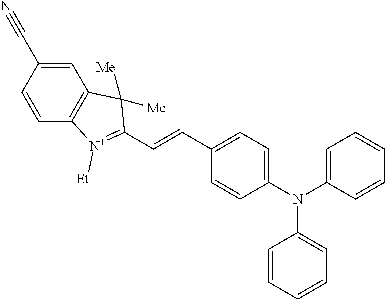 [SMILES: CC[N+]1=C(/C=C/c2ccc(N(c3ccccc3)c3ccccc3)cc2)C(C)(C)c2cc(C#N)ccc21] 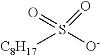 | DY-4 |
| 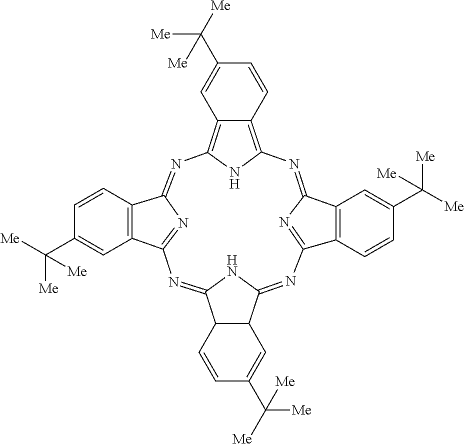 [SMILES: CC(C)(C)C1=CC2c3nc4nc(nc5[nH]c(nc6nc(nc([nH]3)C2C=C1)-c1cc(C(C)(C)C)ccc1-6)c1cc(C(C)(C)C)ccc51)-c1cc(C(C)(C)C)ccc1-4] | DY-5 |
| 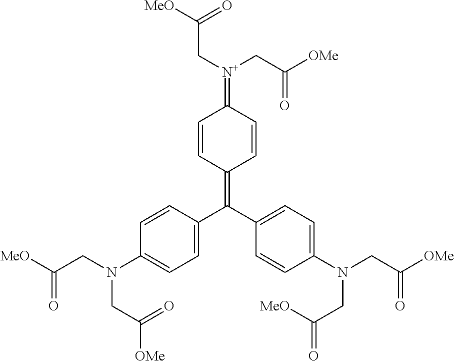 [SMILES: COC(=O)CN(CC(=O)OC)c1ccc(C(=C2C=CC(=[N+](CC(=O)OC)CC(=O)OC)C=C2)c2ccc(N(CC(=O)OC)CC(=O)OC)cc2)cc1] 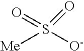 | DY-6 |
| 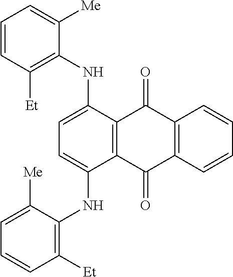 [SMILES: CCc1cccc(C)c1Nc1ccc(Nc2c(C)cccc2CC)c2c1C(=O)c1ccccc1C2=O] | DY-7 |
| 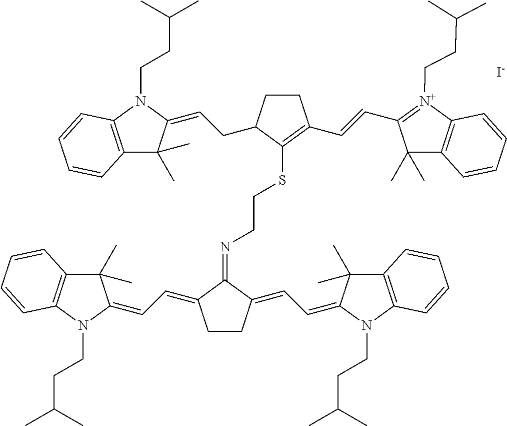 [SMILES: CC(C)CCN1/C(=C/C=C2/CCC(=C\C=C3\N(CCC(C)C)c4ccccc4C3(C)C)/C2=N\CCSC2=C(/C=C/C3=[N+](CCC(C)C)c4ccccc4C3(C)C)CCC2C/C=C2/N(CCC(C)C)c3ccccc3C2(C)C)C(C)(C)c2ccccc21 ; [I-]] | DY-8 |

Besides the ability to convert light into heat, the dyes according general formula I, II, III and the dyes of Table 4 show fluorescence which makes them also suitable for fluorescence medical imaging.

### A.2. The Biocompatible Resin

The biocompatible resin present in the composite resin particle according to the present invention is a resin selected from the group consisting of poly(amino acids), polyphosphazenes, polysaccharide derivatives, poly(esters), poly(ortho esters), poly(cyano-acrylates) and copolymers thereof.

Biodegradable and biocompatible resins can be selected from the resins as discussed in Biodegradable Polymers, PBM Series, Volume 2 (Citus Books 2003, ISSN 1479-1285).

The poly(ester) is preferably a poly(caprolactone), a poly(lactic acid), a poly(L-lactic acid), a poly(glycolic acid), a poly(lactic acid-co-glycolic acid), a poly(L-lactic acid-co-glycolicacid), a poly(D, L-lactide) and any derivative and/or combination thereof.

### A.3. Resin Particle Configuration

The two essential components of the composite resin particle according to the invention, being the dye, preferably the dye having an absorption wavelength between 580 nm and 750 nm, and the resin selected from the group consisting of poly(amino acids), polyphosphazenes, polysaccharide derivatives, poly(esters), poly(ortho esters), poly(cyano-acrylates) and copolymers thereof, can be homogenously or heterogeneously arranged within the resin particle.

A preferable embodiment of the invention, the composite resin particle is a capsule comprising a core and a shell, wherein the core contains the dye, preferably the dye having an absorption wavelength between 580 nm and 750 nm, and the shell comprises the resin selected from the group consisting of poly(amino acids), polyphosphazenes, polysaccharide derivatives, poly(esters), poly(ortho esters), poly(cyano-acrylates) and copolymers thereof. The advantage of encapsulating the dye is that the life time of the dye is increased because the dye is more protected from the aqueous environment avoiding bleaching and hydrolysis of the dye, together with making the dye colloidally stable in the physiological media. In a more preferable embodiment of the invention, the composite resin particle is a capsule wherein the core contains the dye, preferably the dye having an absorption wavelength between 580 nm and 750 nm and the shell comprises a poly(amino acid). The capsule is obtainable by interfacial polymerization of a N-carboxy-anhydride monomer according to general structure IV.

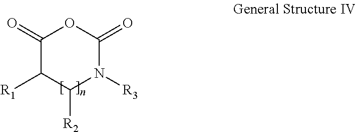

wherein
n represents 0 or 1
R₁, R₂ and R₃ are selected from the group consisting of a hydrogen, a substituted or unsubstituted alkyl group, a substituted or unsubstituted alkenyl group, a substituted or unsubstituted alkynyl group, a substituted or unsubstituted aralkyl group, a substituted or unsubstituted alkaryl group and a substituted or unsubstituted aryl or heteroaryl group
R₁, R₂ and R₃ may represent the necessary atoms to form a five to eight membered ring

In a preferred embodiment n represents 0. In a particular preferred embodiment R₃ represents a hydrogen or an alkyl group, a hydrogen being the most preferred.

In another preferred embodiment R₁ and R₂ are selected from the group consisting of a hydrogen, a substituted or unsubstituted alkyl group, a substituted or unsubstituted aralkyl group, a substituted or unsubstituted alkaryl group and a substituted or unsubstituted aryl group.

In further preferred embodiment, the N-carboxy-anhydride monomer according to general structure is selected from the group consisting of a glycine derivative, an alanine derivative, a leucine derivative, a phenylalanine derivative, a phenylglycine derivative, a valine derivative, a glutamic acid derivative, an aspartic acid derivative, a lysine derivative, an ornithine derivative, a histidine derivative, a methionine derivative, a cysteine derivative, an arginine derivative, a tryptophane derivative, a cysteine derivative, an isoleucine derivative, a tyrosine derivative, a proline derivative and a serine derivative. Both D- and L-amino acid derivatives and mixtures thereof can be used.

The ratio of the weight of the dye to the weight of the resin is preferably from 0.5 wt. % to 10 wt. %, more preferably from 1 wt. % to 5 wt. %. When the weight of the dye is below this range, insufficient heat conversion or fluorescence is obtained. When the weight of the dye is above the above mentioned range, the dye may leak from the core, during the shrinkage process of the core (in the rotary evaporator) or due to not well formed (non-uniform) spheres, some walls are thinner and break or leak dye, forming free dye crystals in the dispersion over time.

The average particle size of the composite resin particles of the invention is preferably from 0.01 μm to 10 μm, more preferably from 0.03 μm to 5 μm and most preferably from 0.03 μm to 3 μm. Particles having an average particle size above these upper limits are difficult to be inserted in the animal or human body via syringes. Particles having an average particle size below these lower limits are difficult to be prepared and partly lose their protective function due to the very large surface area. Other different nano-effects can also occur. Such as agglomeration due to the large surface area, especially in physiological medium later (osmotic pressure, equilibrium mechanisms between the charges of the blood/buffer and the surface of the particle. Biomedically, particles below 200 nm are necessary to cross the blood-brain barrier and enter cells, depending on how the cell wall is constructed, some particles must be even well below 100 nm.

### A.4. Pharmaceutical Active Compound

The composite resin particles according to the invention are also suitable for on-demand drug release wherein the drug is released upon heating the particles by means of an appropriate light source emitting light having a wavelength from 600 to 750 nm. Therefor it is useful to incorporate a pharmaceutical compound to achieve this on-demand drug release.

Sometimes, PTT or PDT cannot completely destruct cancer cells and may result in the survival of the residual cells after photothermal treatment. Therefor it is useful to incorporate anti-cancer drugs for enhanced chemotherapy. The drug will be released upon application of red or NIR light on the composite particle due to the heat generated, triggering synergetic chemo-photothermal therapy. The anti-cancer drug should preferably be soluble in the water immiscible solvent used in the preparation of the composite resin particles (see § B.).

Anti-cancer drugs which are suitable to be incorporated in the particles of the invention are cytostatics. Cytostatics for the treatment of cancer can be selected from the group consisting of alkylating agents, anthracyclines, cytoskeletal disruptors, epothilones, histone deacetylase inhibitors, topoisomerase I inhibitors, topoisomerase II inhibitors, kinase inhibitors, nucleotide analogs, peptide antibiotics, platinum based agents, retinoids and vinca alkaloids and derivatives. Alkylating agents can be bi- or monofunctional. Typical bifunctional alkylating agents are cyclophosphamide, mechlorethamine, chlorambucil and melphalan. Typical monofunctional alkylating agents are dacarbazine, nitrosoureas and temozolomide. Typical anthracyclines are daunorubicin, doxorubicin, epirubicin, idarubicin, mitoxantrone and valrubicin. Typical cytoskeletal disruptors are paclitaxel, docetaxel, abraxane and taxotere. Typical histone deacetylase inhibitors are vorinostat and romidepsin. Typical topoisomerase I inhibitors are irinotecan and topotecan. Typical topoisomerase II inhibitors are etoposide, teniposide and tafluposide. Typical kinase inhibitors are bortezomib, erlotinib, gefitinib, imatinib, vemurafenib and vismodegib. Typical nucleotide analogs are azathioprine, capecitabine, cytarabine, doxifluridine, fluorouracil, gemcitabine, hydroxyurea, mercaptopurine, methotrexate and tioguanine. Typical retinoids are tretinoin, alitretinoin and bexarotene. Typical vinca alkaloids are vinblastine, vincristine and vindesine.

### B. PREPARATION OF COMPOSITE RESIN PARTICLE AND COMPOSITE PARTICLE DISPERSION

The composite resin particle according to the present invention is a mixture of a biocompatible resin which is selected from the group consisting of (poly(amino acids), polyphosphazenes, polysaccharide derivatives, poly(esters), poly(ortho esters), poly(cyano-acrylates) and copolymers thereof, poly(amino acids) and poly(esters) and at least one dye, preferably a dye having an absorption maximum from 580 nm to 750 nm (see § A.1).

The resins according to the present invention can be functionalized with functional groups making the resin self-dispersing in water, with the proviso that the functionalization is limited to avoid that the resin becomes fully water soluble. Typical functional groups making resins self-dispersing are selected from the group consisting of a poly(ethylene oxide), a carboxylic acid or salt thereof, a sulfonic acid or salt thereof, a phosphonic acid or salt thereof, a phosphoric acid monoester or salt thereof, a sulfuric acid monoester or salt thereof, an ammonium group, a sulfonium group and a phosphonium group.

The incorporation of a poly(ethylene glycol) functional group is particularly useful to give stealth properties to the composite resin particles of the invention in the human or animal body. These stealth properties are required to avoid fast uptake by the reticuloendothelial system and only being uphold at the required site.

In a preferred embodiment, the resin according to the present invention is not functionalized with a functional group making the resin self-dispersing.

The use of a dispersant in the preparation of an aqueous dispersion of the composite resin particles is particularly preferred. This allows to adjust the surface characteristics without the need for designing specialty resins. Standard biocompatible resins can be used in the preparation of the resin particles. The dispersant can be a surfactant or a stabilizing polymer. The surfactant can be non-ionic, anionic, cationic or zwitterionic. Non-ionic ethoxylated block copolymer surfactants are preferred. EO-PPO-EO tri-block copolymers even more preferred. The block copolymers give the particles of the invention stealth properties but also lead to smaller particle diameters. As stabilizing polymers, hydroxyl functionalized polymers are particularly preferred, preferably selected from polysaccharides and poly(vinyl alcohol) or poly(vinyl alcohol) copolymers or derivatives thereof. A particularly preferred dispersant is a cationic surfactant comprising at least one primary or secondary amine group and at least one quaternary ammonium group.

A particularly preferred method for the preparation of composite resin particle dispersion according to the present invention includes the following steps:
1. Dissolving the resin according to the present invention and the dye, preferably the dye having an absorption maximum from 580 nm to 750 nm in a water immiscible solvent; and
2. Dissolving a dispersant into water; and
3. Emulsifying the solution of the resin and the dye into the aqueous solution of the dispersant; and
4. Evaporating the water immiscible solvent.

The composite resin particles in dried state if required, can be obtained by isolating the composite particles via a separation technique such as centrifuge, freeze-drying, spray drying, . . . .

If an additional functional compound such as a pharmaceutical active agent has to be integrated in the composite resin particle, this compound is preferably dissolved in the water immiscible solvent. The solvent evaporation technology according to the present invention is particularly of interest for the incorporation of active pharmaceutical ingredients such as anti-cancer drugs.

A particularly preferred method for the preparation of a dispersion of the composite resin particles according to the present invention, comprising an additional functional compound such as an active pharmaceutical ingredient includes the following steps:
1. Dissolving the resin according to the present invention, the dye, preferably the dye having an absorption maximum from 580 nm tot 750 nm and a functional compound in a substantially water immiscible solvent; and
2. Dissolving a dispersant into water; and
3. Emulsifying the solution of the resin, the dye and the functional compound into the aqueous solution of the dispersant; and
4. Evaporating the substantially water immiscible solvent.

In a more preferred embodiment of the invention, the composite resin particles are capsules wherein the core contains the dye, preferably the dye having an absorption maximum from 580 nm tot 750 nm and the shell is a resin selected from the group consisting of poly(amino acids), polyphosphazenes, polysaccharide derivatives, poly(esters), poly(ortho esters), poly(cyano-acrylates) and copolymers thereof. More preferably, the resin is a poly(amino acid) and is prepared using a ring opening polymerization method, an interfacial ring opening polymerization of N-carboxy-anhydrides such as the ones according to general structure IV.

The interfacial polymerization method allows the preparation of capsules in a single step process and over a broad scope of functionalities and particle sizes, making it especially suitable for an industrial production process, more particularly for a continuous industrial process. By simply adjusting the monomer ratios, the technology can easily be tuned towards the functionality to be encapsulated and the physical properties can easily be adjusted towards different applications without major changes in the process conditions leading to a robust technology with considerable latitude towards industrialization.

The ring opening polymerization of N-carboxy-anhydrides has been reviewed by Cheng and Deming (Top. Curr. Chem., 310, 1-26 (2012)). Primary and optionally secondary amines are the most obvious initiators and are widely used to initiate the ring opening polymerization via nucleophilic initiation. Basic initiators can initiate the ring opening polymerization via an activated monomer mechanism, starting by deprotonation of the NCA's followed by ring opening polymerization. When amine initiators are used, both mechanisms often run in parallel. Transition metal initiation is known to give better control on the polymerization. The use of hexamethyldisilazane as initiator has also been disclosed for better controlling the polymerization.

In a further preferred embodiment, a mixture of N-carboxy-anhydrides, derived from different amino acids is used. In an even further embodiment, a mixture of different chirality is used, preferably in a 9/1 to 1/9 ratio of a mixture of D- and L-amino acids. In another preferred embodiment, a mixture of chirality and different amino acids are used. Mixing D- and L-amino acids prevents the poly(amino acid) to form a secondary or tertiary structure as peptides do in nature. The obtained polymeric shell is hence denser and mechanically more resistant. Such a polymeric shell increases the life time of the dye in the human or animal body.

In a particularly preferred interfacial ring opening polymerization method for the preparation of the capsules according to the present invention, the N-carboxy-anhydride monomers and the dye, preferably the dye having an absorption maximum from 580 nm tot 750 nm are dissolved in a substantially water immiscible solvent and emulsified in an aqueous solution containing a polymerization initiator. Upon emulsifying and optionally removing said substantially water immiscible solvent, the ring opening polymerization is initiated at the interface. Upon propagation, a poly(amino acid) shell is formed at the organic-water interface, generating a core-shell structure, encapsulating a functional component or a functional formulation. The obtained polymeric shell is mechanically strong and stable and allows the capsule to be isolated from the liquid wherein the capsules have been prepared.

Suitable N-carboxy-anhydride monomers are described in § A.1, and in Table 1 of the unpublished European Patent Application No. EP21163396.1

The capsules according to the present invention are particularly suited to encapsulate dyes. Micellar based capsules are much less suited to encapsulate dyes. Indeed, the shell of a micellar system is in many cases too permeable with respect to a polymeric shell obtained by the encapsulation method of the invention.

A particularly preferred interfacial ring opening polymerization method comprises the steps of
a) dissolving a compound according to general structure IV and a dye, preferably a dye having an absorption maximum between 580 nm and 750 nm in a water immiscible solvent; and
b) dissolving a polymerization initiator in an aqueous liquid; and
c) emulsifying the solution obtained in step a) into the aqueous liquid; and
d) optionally evaporating the water immiscible solvent; and
e) polymerizing the compound according to general structure IV

The particle size of the capsules of the invention is modified by modifying the emulsification technology, the use of an emulsification aid and the ratio of an emulsification aid to the shell and core during emulsification, the nature of the emulsification aid, changing the viscosity of the continuous or dispersed phase, the ratio of the continuous and dispersed phase, the nature of the core content and the nature of the shell monomers. High shear technologies and ultrasound based technologies are particularly preferred as emulsification technologies. The particle size of the capsules according to the present invention can be tuned by tuning the shear in high shear technologies or by changing the power and amplitude upon sonification.

Di- or multifunctional primary or secondary amines or mixtures thereof are particularly preferred initiators for the ring opening polymerization of the NCA's. The initiators are water soluble and can be functionalized with additional hydrophilic functional groups, preferably selected from the group consisting of a carboxylic acid or salt thereof, a sulfonic acid or salt thereof, a phosphonic acid or salt thereof, a phosphate ester or salt thereof, a sulfate ester or salt thereof, a poly-hydroxyl functionalized group, a poly(ethylene glycol), an ammonium group, a sulfonium group and a phosphonium group.

The incorporation of a poly(ethylene gycol) functional group is particularly useful to give stealth properties to the capsules of the invention if used as drug delivery system in the human or animal body. These stealth properties are required to avoid uptake by the reticuloendothelial system and only release drug at the required site in a controlled manner.

### C. FIELDS OF APPLICATION

The composite resin particles according to the invention are suitable for imaging affected organs in the human and/or animal body. The strong absorption of red or NIR light makes them also suitable for diffuse optical tomography and photoacoustic imaging.

When irradiated with an appropriate red or NIR laser, the dye in the composite particle of the invention can convert the absorbed photon energy into heat, directly ablating cancer cells with minimal invasion to surrounding healthy tissues making the particles very suitable in tumour phototherapy treatment (PTT).

The composite resin particle of the invention is also useful in photodynamic therapy (PDT) wherein the NIR absorber is excited with light of an appropriate wavelength for converting molecular oxygen into cytotoxic reactive oxygen species (ROS), such as singlet oxygen, which in turn damages cancer cells through oxidative stress and consequently induces cell death.

### D. EXAMPLES

### D.1. Materials

D,L-phenylalanine N-carboxy anhydride, D-phenylalanine N-carboxy anhydride and L-phenylalanine N-carboxy anhydride can be prepared according to standard methods as disclosed by Dabashvill et al. (Journal of Physical Chemistry B, 111 (38), 11105-11110 (2007)) and Otake et al. (Angewandte Chemie, International Edition, 57 (35), 11389-11393 (2018)).
L-leucine N-carboxy anhydride, D-leucine N-carboxy anhydride and D,L-leucine N-carboxy anhydride can be prepared according to standard methods as disclosed by Baars et al. (Organic Process Research and Development, 7 (4), 509-513 (2003)).
Crosslinker-1 is a trifunctional beta-keto-ester according to the following structure, which can be prepared as disclosed by Speisschaert et al. (Polymer, 172, 239-246 (2019)).

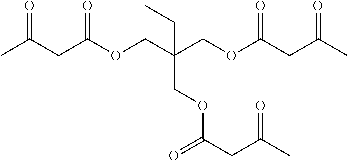 [SMILES: CCC(COC(=O)CC(C)=O)(COC(=O)CC(C)=O)COC(=O)CC(C)=O]

CATSURF-1 is a cationic surfactant according to the following structure, which can be prepared as disclosed in WO2018137993 (Agfa N.V.) as Surf 3.

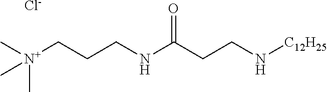

SQ-1 is a dye having its absorption maximum in the range of 580-750 nm and is prepared as follows:
0.11 g of squaric acid was put together with 5 mL pyridine and 0.21 mL (0.23 g) acetic anhydride. The mixture was stirred for one hour. 0.69 g of 1-butyl-2,3,3-trimethyl-3H-Indolium-iodide was added to the mixture and stirred at 60° C. for 30 min. After cooling down to room temperature 20 mL deionized water was added the crude product precipitated and was filtered off. The solid product was washed with water. The washed product was boiled in water, filtered off and dried in vacuum at room temperature. 0.39 g (yield: 77%) of SQ-1 was isolated
SQ-5 is a dye having its absorption maximum in the range of 580-750 nm and is prepared as follows:
0.23 g of squaric acid was put together with 10 mL pyridine and 0.41 mL (0.45 g) acetic anhydride. The mixture was stirred for one hour. 1.43 g of 2,3,3-trimethyl-1-(3-methylbutyl)-3H-Indolium-iodide was added to the mixture and stirred at 60° C. for 30 min. After cooling down to room temperature 15 mL deionized water was added to the reaction mixture. The mixture was stirred overnight. The product crystalized. The crystals were filtered, washed with water and dried in vacuum at room temperature. 0.72 g (yield: 67%) of SQ-5 was isolated.
SQ-6 is a dye having its absorption maximum in the range of 580-750 nm and is prepared as follows:
0.23 g of squaric acid was put together with 10 mL pyridine and 0.41 mL (0.45 g) acetic anhydride. The mixture was stirred for one hour. 1.43 g of 3-butyl-1,1,2-trimethyl-1H-Benz[e]indolium-bromide was added to the mixture and stirred at 110° C. for 30 min. After cooling down 15 mL MeOH and 25 mL deionized water were added to the reaction mixture. The product crystalized partly. Some non-crystalline product was decanted and recrystallize in acetone. The combined crystals were filtered, washed with water and dried in vacuum at room temperature. 0.62 g (yield: 51%) of SQ-6 was isolated
AC-1 is a dye having its absorption maximum in the range of 580-750 nm and is prepared as follows:
3.54 g of starting dye (I) was added to 25 mL MeOH. 1.24 mL (0.98 g) of 2-ethylhexan-1-amine was added and was stirred at 55° C. for 45 min. 0.44 mL (0.56 g) of 2-ethylhexan-1-amine was added and was stirred at 55° C. for another 30 min. 0.32 mL (0.47 g) was added to the mixture and allowed to cool down to room temperature. After the addition of 75 mL deionized water, the mixture was stirred overnight at room temperature. The product crystalized. The crystals were filtered, washed with water and dried in vacuum at room temperature. 3.33 g (yield: 99%) of AC-1 was isolated

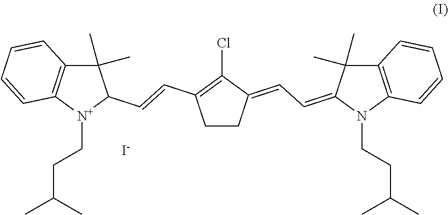 [SMILES: CC(C)CCN1/C(=C/C=C2\CCC(/C=C/C3[NH+](CCC(C)C)c4ccccc4C3(C)C)=C2Cl)C(C)(C)c2ccccc21 ; [I-]]

DYE-1 is a dye having its absorption maximum in the range of 580-750 nm and is prepared as follows:
7.96 g of N-(2-hydroxyphenyl)-2-methoxy-acetamide was dissolved 70.4 mL MeOH and a solution of 10.1 g N4-ethyl-N4-isopropyl-2-methyl-benzene-1,4-diamine and 18.7 g Na2CO3 in 79 mL deionized water and 17 mL MeOH was added. To the mixture a solution of 14.6 g KI and 12 in 12 mL deionized water was added dropwise over a period of 10 min and stirred for 1 h at room temperature. The crude product was filtered of and taken up in 200 ml deionized water (15 min stirring), filtered of again, washed with 40 mL deionized water/MeOH (v/v=9/1) five times and dried at 50° C. The resulting product (15.5 g of 15.9 g) was recrystallized in 100 mL MeOH/deionized water (v/v=95/5), washed with 80 mL MeOH/H2O (v/v=7/3) and dried at 50° C. 11.3 g (yield: 71.5%) of DYE-1 was isolated.
DYE-8 is a dye having its absorption maximum in the range of 580-750 nm and is prepared as follows:
0.50 g of starting dye (I) was added to 20 mL MeOH. 27 mg of 2-aminoethanethiol and 0.1 mL triethylamine was added and was stirred at 65° C. for 1 h and allowed to cool down. After the addition of small amount of deionized water, the mixture was stirred overnight. The product crystalized. The crystals were filtered and dried in vacuum at room temperature. 0.27 g (yield: 59%) of Dye-8 was isolated.

### D.2. Methods

### D.2.1. Particle Size Measurement

The particle size of the capsules was measured using a Zetasizer™ Nano-S (Malvern Instruments, Goffin Meyvis).

D.2.2. Light into Heat Conversion Measurement

The ability of the capsules according to the invention, to convert red laser light into heat is measured as follows: The prepared aqueous capsule dispersions were diluted with water to a final dye concentration of 69 μg/mL in 2 mL and were exposed to an LPC836 laser diode (650 nm) with a power of 350 mW in a polystyrene cuvette.

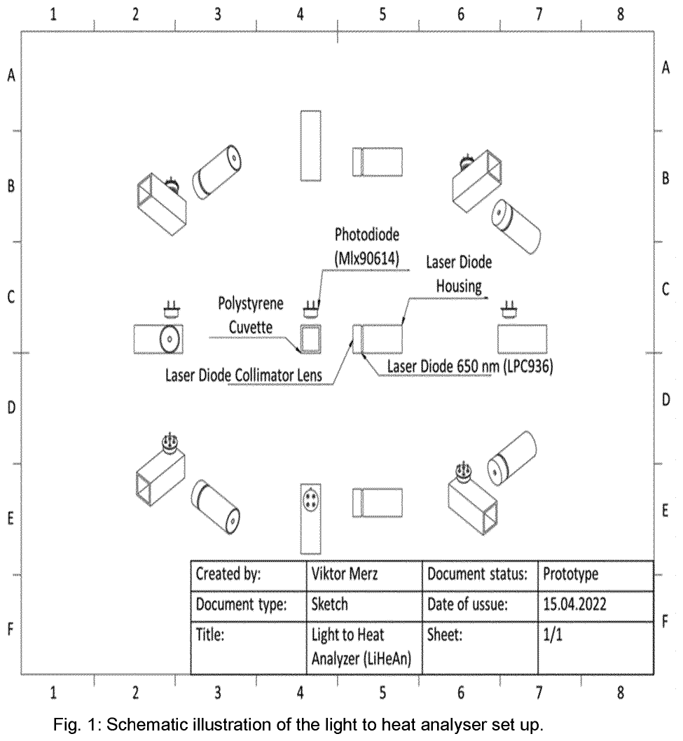

Heat generation by the capsules was measured with a perpendicular infrared thermosensor (RETTI MIx90614Esf) at a distance of 2 mm from the cuvette according to the set up illustrated in FIG. 1.

The temperature increase with respect to the room temperature (ΔT) was determined after 18 minutes of exposure to the laser light.

### D.2.3. Measurement of UV-VIS Spectra of the Dye and the Capsules

Before measuring the UV-VIS spectra of the dyes having an absorption maximum from 580 to 750 nm, the dyes are dissolved in methanol in a concentration of 3.2 μg/mL.

The UV-VIS spectra of the capsules were measured at a dilution of 800 times compared to the prepared dispersion. (see § D.3).

The UV-VIS spectra were measured on an Agilent 8433 spectrophotometer for spectra up to 1100 nm.

### D.2.4. Measurement of Fluorescence Spectra of the Capsules

The fluorescence spectra were measured on with an UV-Vis-NIR spectroradiometer from Ocean Optics HR 4000.

### D.3. Example 1

This example illustrates the ability of the capsules, according to the invention, to convert red laser light into heat.

### Preparation of the Inventive Capsule Dispersions INVCAPS-1 to INVCAPS-6

1.5 g D,L-phenylalanine N-carboxy anhydride, 0.75 g D-leucine N-carboxy anhydride, 0.75 g L-leucine N-carboxy anhydride, 0.34 g Crosslinker-1 and 33 mg of the dye were dissolved in 25 mL ethyl acetate. The mixture was filtered over a 1.7-micron filter.

The type of the dye dissolved is listed in Table 5.

A second solution was prepared by dissolving 0.6 g surfactant CATSURF-1 in 30 ml water.

The first solution was added to the second and mixed with an Ultra Turrax T25 for 5 min at 15000 RPM while maintaining the temperature at 20-30° C. The solvent was removed under reduced pressure to a total weight of 30 g. The polymerization was allowed to continue at room temperature for 24 hours.

The average particle size of the obtained capsule dispersions was measured according to D.2.1. and the values are listed in Table 5. The absorption maximum of the dye was measured according to § D.2.3.

TABLE 5

| Caps | | Average particle | λmax of dye in 580 |
|---|---|---|---|
| dispersion | Dye | size (nm) | to 750 nm range |
| INVCAPS-1 | SQ-1 | 192 | 662 |
| INVCAPS-2 | SQ-5 | 146 | 656 |
| INVCAPS-3 | SQ-6 | 132 | 662 |
| INVCAPS-4 | AC-1 | 135 | 656 |
| INVCAPS-5 | Dye-1 | 171 | 660 |
| INVCAPS-6 | Dye-8 | 141 | 650 |
| COMPCAPS | — | 195 | — |

### Preparation of the Comparative Capsule Dispersion COMPCAPS

The comparative capsule dispersion was obtained the same way as the inventive capsule dispersion, but without the presence of the dye (see Table 5)

### Light to Heat Conversion

The laser light to heat conversion ability of the inventive capsule dispersions and the comparative capsule dispersion was measured as described in D.2.2. The results a listed in Table 6.

TABLE 6

| Caps | | λmax of | Fluorescence |
|---|---|---|---|
| dispersion | ΔT (° C.) | dispersion | (λmax) [nm] |
| INVCAPS-1 | 5.3 | 675 | 630-830 (675) |
| INVCAPS-2 | 7.3 | 643 | 650-820 (660) |
| INVCAPS-3 | 9.0 | 662 | 790-870 (820) |
| INVCAPS-4 | 6.6 | 660 | 790-860 (830) |
| INVCAPS-6 | 11.8 | 666 | — |
| COMPCAPS | 1.6 | — | 630-680 (646)* |

* The observed fluorescence is extremely low and is probably due to impurities.

From the results of Table 6, it can be concluded that the capsules comprising the dyes having an absorption maximum from 580 nm to 750 nm an a polymeric shell comprising poly(amino acid) show an ability to convert red laser light into heat, leading to a much higher temperature increase than the same capsule without a dye.

The inventive capsules show an ability to convert laser light into heat making them useful in photothermal therapy (PTT), photodynamic therapy (PDT), chemo-photodynamic therapy.

From the results of Table 6, it can also be concluded that some of the inventive capsule as described above show fluorescence making them useful for fluorescence medical imaging.

1-17. (canceled)

18. A composite resin particle comprising a dye and a resin selected from the group consisting of poly(amino acids), polyphosphazenes, polysaccharide derivatives, poly(esters), poly(ortho esters), poly(cyano-acrylates), and copolymers thereof, characterized in that the composite resin particle has an absorption maximum in a range from 600 nm to 750 nm, wherein the absorption maximum is measured on an aqueous dispersion of the composite resin particle at a concentration of 0.19 mg/mL, and further characterized in that the average particle size of the particles is between 0.03 μm and 3 μm.

19. The composite resin particle of claim 18, wherein the dye is selected from the group consisting of azo-dyes, phtalocyananines, anthraquinones, indigoid dyes, cyanines, merocyanines, oxonoles, triaryl methane dyes, azomethine dyes, and indoaniline dyes.

20. The composite resin particle of claim 19, wherein the ratio of the weight of the dye to the weight of the resin is from 1 wt. % to 5 wt. %.

21. The composite resin particle of claim 19, wherein the cyanine is a squarilium dye according to general formula I: 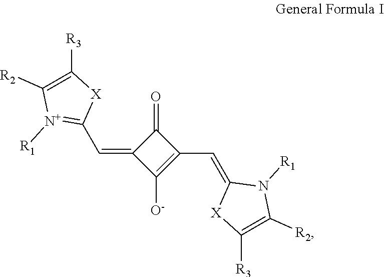
wherein
R₁ is selected from the group consisting of a substituted or unsubstituted alkyl group, a substituted or unsubstituted alkenyl group, a substituted or unsubstituted alkynyl group, a substituted or unsubstituted alkaryl group, a substituted or unsubstituted aralkyl group, and a substituted or unsubstituted aryl or heteroaryl group;
R₂ and R₃ are each independently selected from the group consisting of a hydrogen, a substituted or unsubstituted alkyl group, a substituted or unsubstituted alkenyl group, a substituted or unsubstituted alkynyl group, a substituted or unsubstituted alkaryl group, a substituted or unsubstituted aralkyl group, a substituted or unsubstituted aryl or heteroaryl group, a halogen, an ester, an amide, an ether, a nitro, a nitrile, an amine, a ketone, and an aldehyde;
R₂ and R₃ may represent the necessary atoms to form a five or six membered ring;
X is selected from the group consisting of S, O, NR₄, and CR₅R₆;
R₄, R₅, and R₆ are each independently selected from the group consisting of a substituted or unsubstituted alkyl group, a substituted or unsubstituted alkenyl group, a substituted or unsubstituted alkynyl group a substituted or unsubstituted alkaryl group, a substituted or unsubstituted aralkyl group, and a substituted or unsubstituted aryl or heteroaryl group; and
R₅ and R₆ may represent the necessary atoms to form a five to eight membered ring.

22. The composite resin particle of claim 19, wherein the cyanine dye is a pentamethine cyanine dye according to general formula II: 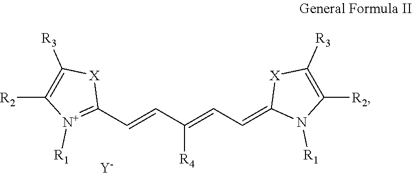
wherein
R₁ is selected from the group consisting of a substituted or unsubstituted alkyl group, a substituted or unsubstituted alkenyl group, a substituted or unsubstituted alkynyl group a substituted or unsubstituted alkaryl group, a substituted or unsubstituted aralkyl group, and a substituted or unsubstituted aryl or heteroaryl group;
R₂ and R₃ are each independently selected from the group consisting of a hydrogen, a substituted or unsubstituted alkyl group, a substituted or unsubstituted alkenyl group, a substituted or unsubstituted alkynyl group, a substituted or unsubstituted alkaryl group, a substituted or unsubstituted aralkyl group, a substituted or unsubstituted aryl or heteroaryl group, a halogen, an ester, an amide, an ether, a nitro, a nitrile, an amine, a ketone, and an aldehyde;
R₂ and R₃ may represent the necessary atoms to form a five or six membered ring;
R₄ is selected from the group consisting of a hydrogen, a substituted or unsubstituted alkenyl group, a substituted or unsubstituted alkynyl group a substituted or unsubstituted alkaryl group, a substituted or unsubstituted aralkyl group, and a substituted or unsubstituted aryl or heteroaryl group;
X is selected from the group consisting of S, O, NR₅, and CR₆R₇;
R₅, R₆, and R₇ are each independently selected from the group consisting of a substituted or unsubstituted alkyl group, a substituted or unsubstituted alkenyl group, a substituted or unsubstituted alkynyl group a substituted or unsubstituted alkaryl group, a substituted or unsubstituted aralkyl group, and a substituted or unsubstituted aryl or heteroaryl group; and
R₆ and R₇ may represent the necessary atoms to form a five to eight membered ring.

23. The composite resin particle of claim 19, wherein the cyanine dye is a cyanine dye according to general formula III: 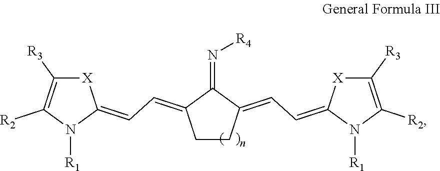
wherein
R₁ is selected from the group consisting of a substituted or unsubstituted alkyl group, a substituted or unsubstituted alkenyl group, a substituted or unsubstituted alkynyl group a substituted or unsubstituted alkaryl group, a substituted or unsubstituted aralkyl group, and a substituted or unsubstituted aryl or heteroaryl group;
R₂ and R₅ are each independently selected from the group consisting of a hydrogen, a substituted or unsubstituted alkyl group, a substituted or unsubstituted alkenyl group, a substituted or unsubstituted alkynyl group, a substituted or unsubstituted alkaryl group, a substituted or unsubstituted aralkyl group, a substituted or unsubstituted aryl or heteroaryl group, a halogen, an ester, an amide, an ether, a nitro, a nitrile, an amine, a ketone, and an aldehyde;
R₂ and R₃ may represent the necessary atoms to form a five or six membered ring;
R₄ is selected from the group consisting of a substituted or unsubstituted alkyl group, a substituted or unsubstituted alkenyl group, a substituted or unsubstituted alkynyl group, a substituted or unsubstituted alkaryl group, a substituted or unsubstituted aralkyl group and a substituted or unsubstituted aryl or heteroaryl group;
X is selected from the group consisting of S, O, NR₅, and CR₆R₇;
R₅, R₆, and R₇ are each independently selected from the group consisting of a substituted or unsubstituted alkyl group, a substituted or unsubstituted alkenyl group, a substituted or unsubstituted alkynyl group, a substituted or unsubstituted alkaryl group, a substituted or unsubstituted aralkyl group, and a substituted or unsubstituted aryl or heteroaryl group; and
R₆ and R₇ may represent the necessary atoms to form a five to eight membered ring.

24. The composite resin particle of claim 18, wherein the particle is a capsule consisting of a polymeric shell surrounding a core, the core comprises the dye, the polymeric shell comprises a poly(amino acid) and is obtainable by interfacial polymerization of a N-carboxy-anhydride monomer according to general structure IV: 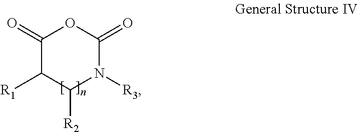
wherein
n represents 0 or 1;
R₁, R₂, and R₅ are each independently selected from the group consisting of a hydrogen, a substituted or unsubstituted alkyl group, a substituted or unsubstituted alkenyl group, a substituted or unsubstituted alkynyl group, a substituted or unsubstituted aralkyl group, a substituted or unsubstituted alkaryl group, and a substituted or unsubstituted aryl or heteroaryl group; and
R₁, R₂, and R₃ may represent the necessary atoms to form a five to eight membered ring.

25. The composite resin particle of claim 19, wherein the particle is a capsule consisting of a polymeric shell surrounding a core, the core comprises the dye, the polymeric shell comprises a poly(amino acid) and is obtainable by interfacial polymerization of a N-carboxy-anhydride monomer according to general structure IV: 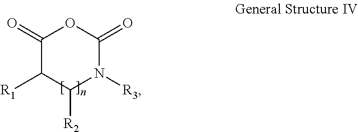
wherein
n represents 0 or 1;
R₁, R₂, and R₃ are each independently selected from the group consisting of a hydrogen, a substituted or unsubstituted alkyl group, a substituted or unsubstituted alkenyl group, a substituted or unsubstituted alkynyl group, a substituted or unsubstituted aralkyl group, a substituted or unsubstituted alkaryl group and a substituted or unsubstituted aryl or heteroaryl group; and
R₁, R₂, and R₃ may represent the necessary atoms to form a five to eight membered ring.

26. The composite resin particle of claim 25, wherein the polymeric shell comprises a crosslinker.

27. The composite resin particle of claim 26, further comprising a pharmaceutical active compound.

28. The composite resin particle of claim 27, wherein the pharmaceutical active compound is a cytostatics selected from the group consisting of alkylating agents, anthracyclines, cytoskeletal disruptors, epothilones, histone deacetylase inhibitors, topoisomerase I inhibitors, topoisomerase II inhibitors, kinase inhibitors, nucleotide analogs, peptide antibiotics, platinum based agents, retinoids and vinca alkaloids, and derivatives thereof.

29. An aqueous dispersion comprising the composite resin particles as defined in claim 18 and a stabilizing polymer or a surfactant.

30. An aqueous dispersion comprising the composite resin particles as defined in claim 19 and a stabilizing polymer or a surfactant.

31. The aqueous dispersion of claim 29, wherein the surfactant is a cationic surfactant comprising at least one primary or secondary amine group and at least one quaternary ammonium group.

32. A pharmaceutical composition comprising the dispersion as defined in claim 29 and a pharmaceutical carrier or excipient.

33. A pharmaceutical composition comprising the dispersion as defined in claim 30 and a pharmaceutical carrier or excipient.

34. A pharmaceutical composition comprising the dispersion as defined in claim 29 for use in cancer therapy.

35. A pharmaceutical composition comprising the dispersion as defined in claim 29 for use in a medical imaging method.

36. A method of preparing an aqueous dispersion as defined in claim 29 comprising the steps of:
a) dissolving a resin selected from the group consisting of (poly(amino acids), polyphosphazenes, polysaccahride derivatives, poly(esters), poly(ortho esters), poly(cyano-acrylates), and copolymers thereof and a dye having an absorption maximum between 580 and 750 nm in a substantially water immiscible solvent;
b) dissolving a dispersant into water;
c) emulsifying the solution of the resin and the dye obtained in step a) into the aqueous solution of the dispersant obtained in step b); and
d) evaporating the substantially water immiscible solvent.

37. A method of preparing the aqueous dispersion as defined in claim 29 comprising the steps of:
a) dissolving a N-carboxy-anhydride monomer according to general structure IV and a dye having an absorption maximum from 580 nm to 750 nm in a water immiscible solvent;
b) dissolving a polymerization initiator in an aqueous liquid;
c) emulsifying the solution obtained in step a) into the aqueous liquid;
d) optionally evaporating the water immiscible solvent; and
e) polymerizing the N-carboxy-anhydride monomer according to general structure IV.
